(19) United States
(12) Patent Application Publication
(10) Pub. No.: US 2006/0047299 A1
(43) Pub. Date: Mar. 2, 2006

(54) Vascular occlusive wire with extruded bioabsorbable sheath

(76) Inventor: Patrick J. Ferguson, Portland, OR (US)

Correspondence Address: GLENN C. BROWN, PC, 777 NW WALL STREET, SUITE 308, BEND, OR, 97701, US

(21) Appl. No.: 10/925,782

(22) Filed: Aug. 24, 2004

(51) Int. Cl. A61M 29/00

(52) U.S. Cl. 606/200

(57) ABSTRACT

A vascular occlusive wire having an extruded bioabsorbable sheath. The sheath is preferably formed from a copolymeric material, and in one embodiment is extruded in two steps.

### BACKGROUND OF THE INVENTION

The art and science of interventional therapy and surgery has continually progressed towards treatment of internal defects and diseases by use of ever smaller incisions or access through the vasculature or body openings in order to reduce the trauma to tissue surrounding the treatment site. One important aspect of such treatments involves the use of catheters to place therapeutic devices at a treatment site by access through the vasculature. Examples of such procedures include transluminal angioplasty, placement of stents to reinforce the walls of a blood vessel or the like and the use of vasoocclusive devices to treat defects in the vasculature. There is a constant drive by those practicing in the art to develop new and more capable systems for such applications. When coupled with developments in biological treatment capabilities, there is an expanding need for technologies that enhance the performance of interventional therapeutic devices and systems.

One specific field of interventional therapy that has been able to advantageously use recent developments in technology is the treatment of neurovascular defects. More specifically, as smaller and more capable structures and materials have been developed, treatment of vascular defects in the human brain which were previously untreatable or represented unacceptable risks via conventional surgery have become amenable to treatment. One type of non-surgical therapy that has become advantageous for the treatment of defects in the neurovasculature has been the placement by way of a catheter of vasoocclusive devices in a damaged portion of a vein or artery.

Vasoocclusion devices are therapeutic devices that are placed within the vasculature of the human body, typically via a catheter, either to block the flow of blood through a vessel making up that portion of the vasculature through the formation of an embolus or to form such an embolus within an aneurysm stemming from the vessel. The vasoocclusive devices can take a variety of configurations, and are generally formed of one or more elements that are larger in the deployed configuration than when they are within the delivery catheter prior to placement. One widely used vasoocclusive device is a helical wire coil having a deployed configuration which may be dimensioned to engage the walls of the vessels. One anatomically shaped vasoocclusive device that forms itself into a shape of an anatomical cavity such as an aneurysm and is made of a pre-formed strand of flexible material that can be a nickel-titanium alloy is known from U.S. Pat. No. 5,645,558, which is specifically incorporated by reference herein. That vasoocclusive device comprises one or more vasoocclusive members wound to form a generally spherical or ovoid shape in a relaxed state. The vasoocclusive members can be a helically wound coil or a co-woven braid formed of a biocompatible material, and the device is sized and shaped to fit within a vascular cavity or vesicle, such as for treatment of an aneurysm or fistula. The vasoocclusive member can be first helically wound or braided in a generally linear fashion, and is then wound around an appropriately shaped mandrel or form, and heat treated to retain the shape after removal from the heating form.

The delivery of such vasoocclusive devices can be accomplished by a variety of means, including via a catheter in which the device is pushed through the catheter by a pusher to deploy the device. The vasoocclusive devices, which can have a primary shape of a coil of wire that is then formed into a more complex secondary shape, can be produced in such a way that they will pass through the lumen of a catheter in a linear shape and take on a complex shape as originally formed after being deployed into the area of interest, such as an aneurysm. A variety of detachment mechanisms to release the device from the pusher are known in the art.

Once in place in the aneurysm, the vasoocclusive coil triggers a response in the body by which tissue is deposited over and around the coil. Disruption and stagnation of the blood flow by the vasoocclusive coil triggers intra-aneurysmal thrombus formation. Endothelial cells originating from the parent artery migrate over the thrombus, covering the aneurysm neck. Leukocytes trapped within the thrombus begin to ingest platelets, red blood cells and fibrin through the process of phagocytosis. Leukocytes continue to infiltrate aneurismal thrumbus and the thrombus is transformed into myofibroblasts, or smooth muscle cells. The smooth muscle cells in the aneurysm begin to secrete collagen. Smooth muscle cells within a collagen network comprise fibro-cellular tissue. Through thrombus organization, the aneurysm sac is filled with fibro-cellular tissue promoting stability of the aneurysm sac. The tissue formation eventually occludes the aneurysm, forming a “patch” on the vascular wall and isolating the aneurysm.

In one method of promoting the biological response to the coil, a bioabsorbable suture material is tightly wound around the portion of the coil that is to be inserted into the aneurysm. The suture is tightly wound onto the coil, and the assembly is then heat cured at a relatively low temperature to fuse the suture windings together. The bioabsorbable material promotes the body's tissue building response, and results in a predictable and desirable rate of occlusion of the aneurysm. While this method of encasing the coil in bioabsorbable achieves the goal of promoting the body's tissue response, the process of tightly winding and curing the suture is relatively complicated and expensive.

A need therefore remains for a simpler and less expensive method of coating the distal portion of the vasoocclusive wire assembly so that it still provides the advantages of a bioabsorbable covering but which can be accomplished more efficiently.

### BRIEF DESCRIPTION OF THE DRAWINGS

FIG. 1 is a cross-sectional view of a vascoocclusive wire according to the invention being inserted into a typical aneurysm.

FIG. 2 a cross-sectional view of a plurality of vascoocclusive wires according to the invention disposed in a typical aneurysm, and showing an additional vascoocclusive wire being inserted into the aneurysm.

FIG. 3 is a side view of a coiled vasoocclusive wire according to the prior art, and which is covered with a spiral-wound suture material.

FIG. 4 is an enlarged side view of the vasoocclusive wire shown in FIG. 3.

FIG. 5 is a cross-sectional view of the vasoocclusive wire shown in FIG. 3.

FIG. 6 is a side view of a coiled vasoocclusive wire according to the present invention, including an outer sheath formed of extruded suture material.

FIG. 7 is an enlarged side view of the vasoocclusive wire shown in FIG. 6.

FIG. 8 is a cross-sectional view of the vasoocclusive wire shown in FIG. 6.

### DETAILED DESCRIPTION OF THE INVENTION

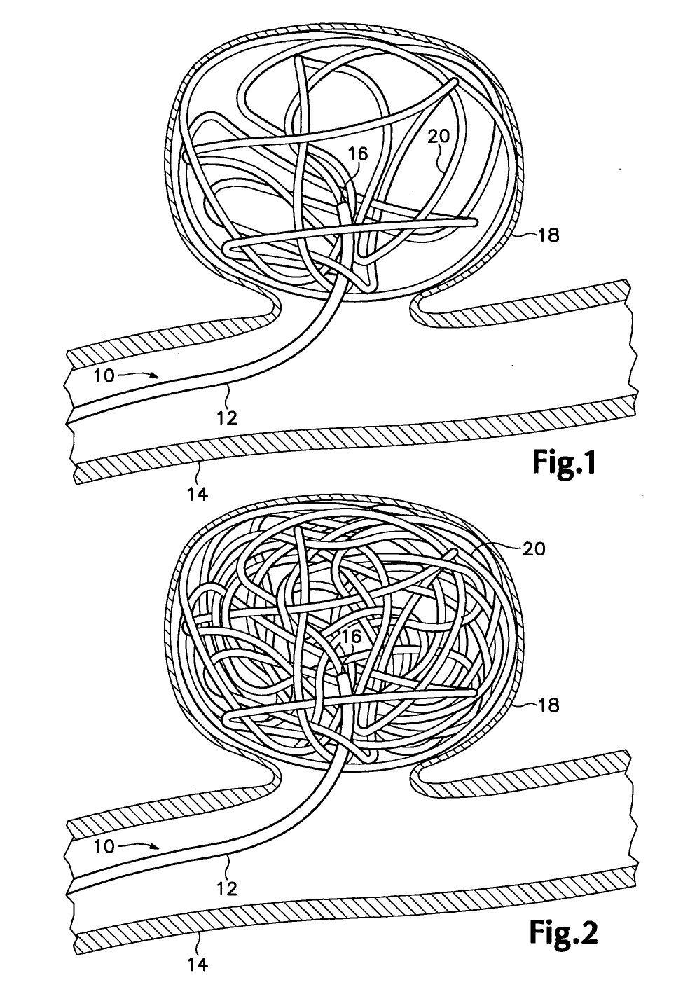

Turning now to FIGS. 1 and 2, a vascooclusive device is shown generally at 10, and includes a catheter 12 and a vasooclusive wire 20. Catheter 10 has been inserted into an artery 14, and its distal end 16 positioned in the opening of an aneurysm 18. A first vasoocclusive wire 20 is inserted into the catheter 12, and emerges from catheter end 16 and inserts into aneurysm 18. Once inserted into the aneurysm, a distal portion of wire 20 is severed and remains in the aneurysm. In a typical procedure, a number of vasoocclusive wires, as many as 15-20 in many instances, are inserted in order to adequately fill the aneurysm. An aneurysm having multiple vasoocclusive wires is shown in FIG. 2.

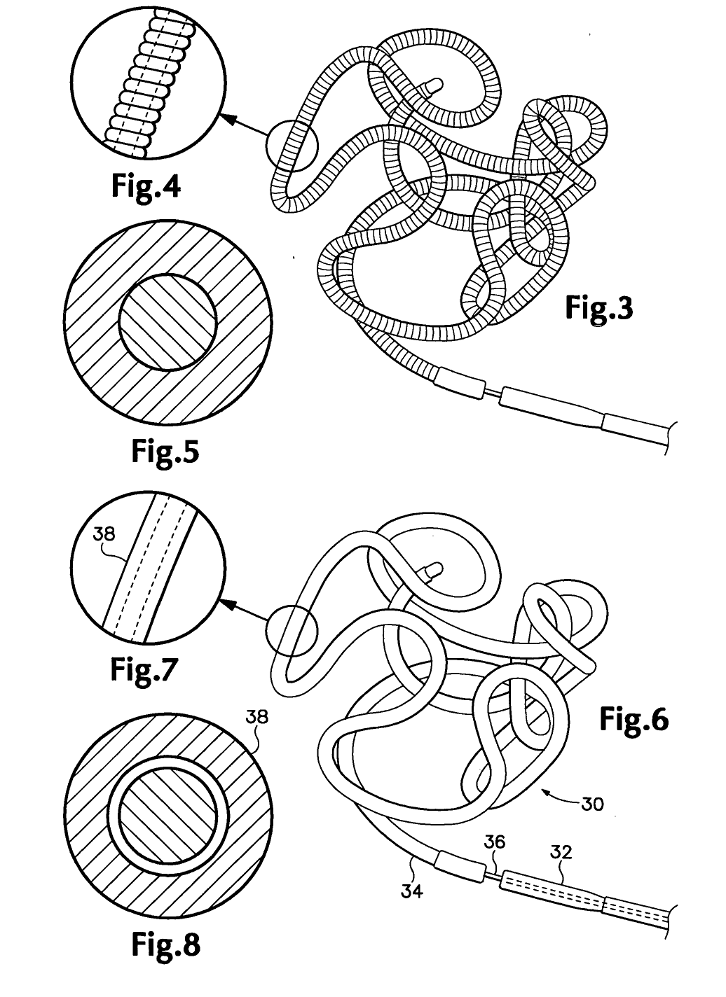

Referring now to FIGS. 3-5 a vasoocclusive wire according to a preferred embodiment of this invention is shown at 30. Wire 30 includes a base portion 32, a distal portion 34, and a severable portion 36 linking the base portion 32 and distal portion 34. Distal portion 32 is preferably formed of platinum or a platinum alloy, and has been treated according to well-known techniques to naturally assume a convoluted shape such as that shown in FIG. 3. Most such techniques involve using a mandrel to form the distal wire portion into the desired shape, and then heat treating to “set” the distal portion into the desired shape. The resilient properties of the material permit the wire to be temporarily straightened under a relatively light tensile load, and to resume the desired shape when released.

Either before or after being treated as described, distal portion 34 is attached to base portion 32 by a severable portion 36 according to well-known techniques. In one such technique, severable portion 36 is formed of a low-melting, conductive metal material that can be remotely severed by passing a small electrical current through the device. The low-melting conductive metal has a sufficient electrical resistance to heat the severable portion to its melting temperature, thereby severing and releasing distal portion 34 from base portion 32. In another embodiment a single wire is used, and the distal portion is rendered severable by swaging or deforming the wire at the severing point to form a high resistance portion that rapidly heats to its melting point when a current is passed through the wire.

Referring to FIGS. 4 and 5, in one novel aspect of the invention distal portion 34 is encased in an extruded sheath 38 of bioabsorbable material, such as a 90:10 polyglycolic acid/polydiaxanone copolymer. Sheath 38 is formed by being extruded using known extrusion techniques applicable to fine suture material. In one preferred embodiment, the suture material is extruded in a two step process. It is first extruded into a hollow sheath configuration using a Killian or equivalent polymer extruder to an intermediate outer diameter and wall thickness. The inner diameter of sheath 38 is fixed by the mandrel diameter, and is selected to closely receive distal portion 34 of wire 30. The sheath and is then extruded a second time to a smaller outer diameter and wall thickness. In one preferred embodiment the final outer diameter is equivalent to that of a 9/0 suture. The sheath material is then heat treated by being wound onto a polycarbonate reel, and placed in an autoclave in a moisture-free, oxygen-free, nitrogen atmosphere for 12-25 hours at a temperature of about 136 degrees Celsius. The heat treatment reduces the monomer/polymer ratio and increases the strength of the sheath.

Those of skill in the art will appreciate that other suture materials could be substituted, and that the invention is not limited to any specific sheathing material. The use of an extruded bioabsorbable sheath represents a significant improvement over the prior art in that it can be produced more simply and more economically than a spiral wound sheath, while at the same time providing equivalent or even better absorption properties compared to spiral-wound sheaths.

What is claimed is:

1. A vascular occlusive wire assembly comprising:
a wire having a base portion and a detachable distal portion; and,
the distal wire portion comprising an inner core disposed within an extruded, bioabsorbable outer sheath.

2. A vascular occlusive wire assembly according to claim 1 further comprising:
the vascular cannula operable to position the detachable distal wire portion adjacent a predetermined vascular opening; and,
the wire operable to pass the detachable distal wire portion through the cannula and into the vascular opening.

3. A vascular occlusive wire assembly according to claim 1 further comprising the outer sheath being formed by extruding a bioabsorbable material a first time to a first outer diameter, and then extruding the bioabsorbable material a second time to a second outer diameter.

4. A vascular occlusive wire assembly according to claim 1 wherein the biabsorbable material comprises a polymer.

5. A vascular occlusive wire assembly according to claim 1 wherein the biabsorbable material comprises a copolymer.

6. A vascular occlusive wire assembly according to claim 1 wherein the biabsorbable material comprises polyglycolic acid.

7. A vascular occlusive wire assembly according to claim 1 wherein the biabsorbable material comprises polydiaxanone.

8. A vascular occlusive wire assembly according to claim 1 wherein the biabsorbable material comprises a polyglycolic acid/polydiaxanone copolymer.

9. A vascular occlusive wire assembly according to claim 1 wherein the biabsorbable material comprises a 90:10 polyglycolic acid/polydiaxanone copolymer.

10. A vascular occlusive wire assembly according to claim 1 wherein the distal wire portion comprises a resilient wire operable between a first convoluted configuration and a second extended configuration.

11. A vascular occlusive wire assembly according to claim 1 wherein the distal portion is severable from the base portion.

12. A vascular occlusive wire assembly according to claim 1 wherein the distal portion is remotely severable from the base portion.
